(19) United States
(12) Patent Application Publication
(10) Pub. No.: US 2026/0364238 A1
(43) Pub. Date: Nov. 19, 2026

(54) DOUBLE PERITONEAL DIALYSIS CATHETER AND SHIELD

(71) Applicant: BOARD OF REGENTS, THE UNIVERSITY OF TEXAS SYSTEM, Austin, TX (US)

(72) Inventor: Luca Cicalese, Austin, TX (US)

(21) Appl. No.: 19/678,887

(22) Filed: May 15, 2026

Related U.S. Application Data
(60) Provisional application No. 63/806,217, filed on May 15, 2025.

(51) Int. Cl. A61M 1/28; A61M 25/00; A61M 25/10

(52) U.S. Cl. CPC A61M 1/285; A61M 25/0071; A61M 2025/1004; A61M 2025/1061; A61M 2205/16

(57) ABSTRACT

Various examples are provided related to double peritoneal dialysis catheters and peritoneal dialysis catheter shields. In one example, the catheter device can include a split tubing having a distal end, a plurality of proximal ends including a first proximal end and a second proximal end, and a connection point between the distal end and the plurality of proximal ends. The split tubing can further have a primary lumen extending axially through the split tubing from the distal end to the connection point, a first secondary lumen extending axially through the split tubing from the connection point to the first proximal end of the split tubing, and a second secondary lumen extending axially through the split tubing from the connection point to the second proximal end of the split tubing. A shield can be configured to engage the external surface of the device.

### CROSS REFERENCE TO RELATED APPLICATIONS

This application claims priority to, and the benefit of, U.S. provisional application entitled “Double Peritoneal Dialysis Catheter and Shield” having Ser. No. 63/806,217, filed May 15, 2025, which is hereby incorporated by reference in its entirety.

### BACKGROUND

Peritoneal dialysis (PD) is a home-based renal replacement therapy for patients with end stage renal disease (ESRD). The PD process works by allowing dialysate fluid to flow into the peritoneal cavity via a catheter, where waste products from the blood are filtered and drained out. Because it can be performed in the patient's home, PD allows relatively more freedom and requires fewer dietary restrictions. However, catheter occlusion is often caused by the greater omentum, which is a common issue leading to dialysate fluid exchange failure. This can lead to at least two additional surgical procedures, one to remove the malfunctioning PD catheter and one to reinsert a new one. Single-lumen dialysis catheters can be particularly susceptible to occlusion by the omentum, negatively impacting patient outcomes and increasing healthcare costs.

### SUMMARY

Aspects of the present disclosure are related to double peritoneal dialysis catheters and shields for peritoneal dialysis catheters. In one aspect, among others, a catheter device can have a split tubing having a distal end, a plurality of proximal ends including a first proximal end and a second proximal end, and a connection point between the distal end and the plurality of proximal ends. The split tubing can further have a primary lumen extending axially through the split tubing from the distal end to the connection point, a first secondary lumen extending axially through the split tubing from the connection point to the first proximal end of the split tubing, a second secondary lumen extending axially through the split tubing from the connection point to the second proximal end of the split tubing, and one or more openings disposed along an external surface of the first and second proximal ends of the split tubing. Each of the one or more openings can extend transversely through a wall of the split tubing and be in fluidic communication with the first and second secondary lumens, and wherein the first and second secondary lumens are in fluidic communication with the primary lumen of the split tubing at the connection point.

In one or more aspects, a diameter of the primary lumen can be greater than a diameter of the first secondary lumen and a diameter of the second secondary lumen. The first and second proximal ends can be coiled. The split tubing can further have one or more Dacron cuffs surrounding an exterior surface of the split tubing and configured to engage a tissue of a user. The distal end of the split tubing can have a connector configured to engage one or more fluid reservoirs. The catheter device can have one or more shields configured to engage the external surface of the split tubing at a point between the connection point and the distal end, or at a point between the connection point and the first proximal end or the second proximal end, and extend radially outward from the external surface of the split tubing.

In another aspect, a catheter system can have a tubing having an internal lumen extending axially through the tubing, a proximal end, a distal end, and one or more openings disposed along an external surface of the proximal end, wherein each of the one or more openings extends transversely through a wall of the tubing and is in fluidic communication with the internal lumen. In one or more aspects, a shield can be configured to engage the external surface of the tubing at a point between the proximal end and the distal end and extend radially outward from the tubing. The distal end of the tubing can have a connector configured to engage one or more fluid reservoirs or a second tubing. The proximal end of the tubing can be coiled. The tubing can further have one or more Dacron cuffs surrounding an exterior surface of the tubing and configured to engage a tissue of a user. The shield can be a disk shape. The shield can be an inflatable donut shaped balloon and the tubing can further have an inflation channel extending axially from the distal end through the tubing to the balloon and adjacent to the internal lumen, wherein the inflation channel is in fluidic communication with the balloon. The shield can be a collapsible umbrella shield configured to remain collapsed along the exterior surface of the tubing when introduced in an abdomen of a user and to open away from the exterior surface of the tubing when the tubing is positioned within the abdomen of the user.

In another aspect, a method can involve introducing a catheter into an abdominal cavity of a patient, the catheter having a tubing, an internal lumen extending axially through the tubing, a proximal end, a distal end, and one or more openings disposed along an external surface of the proximal end, wherein each of the one or more openings extends transversely through a wall of the tubing and is in fluidic communication with the internal lumen, and a shield configured to engage the external surface of the tubing at a point between the proximal end and the distal end and extend radially outward from the tubing, wherein the shield is an inflatable donut shaped balloon and the tubing further having an inflation channel extending axially from the distal end through the tubing to the balloon and adjacent to the internal lumen, wherein the inflation channel is in fluidic communication with the balloon. The method can further involve positioning the catheter within the Douglas pouch of the abdominal cavity of the patient and inflating the balloon by injecting a fluid into the inflation channel. In one or more aspects, the method can further involve deflating the balloon by withdrawing the fluid from the inflation channel and removing the catheter from the abdominal cavity of the patient. The distal end of the tubing can have a connector configured to engage one or more fluid reservoirs or a second tubing. The method can further involve connecting a first fluid reservoir comprising a dialysis solution to the connector, introducing the dialysis solution into the abdominal cavity through the catheter, and disconnecting the first fluid reservoir from the connector. The method can further involve connecting a second fluid reservoir to the connector, draining a waste fluid from the abdominal cavity through the catheter into the second fluid reservoir, and disconnecting the second fluid reservoir from the connector. The tubing can further have one or more Dacron cuffs surrounding an exterior surface of the tubing and configured to engage a tissue of the patient. The tubing can have latex, silicone, polyvinyl chloride (PVC), rubber, or a combination of any thereof.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B are images showing a double peritoneal dialysis (PD) catheter prototype according to one embodiment of the present disclosure.

FIGS. 2A-2C are images showing a setup of an experiment according to one embodiment of the present disclosure.

FIG. 3 is a graph showing an example of the results (mean of 15 measurements each) of an experiment according to one embodiment of the present disclosure.

FIG. 4 is a pair of images showing a simulated catheter flow restriction using an actual human greater omentum obtained from a cadaver according to one embodiment of the present disclosure.

FIGS. 5A-5C are images showing a shield incorporated in a catheter according to one embodiment of the present disclosure. Illustrated are 3 different sizes of this shield (10 cm diameter in FIG. 5A, 8 cm diameter in FIG. 5B, and 6 cm diameter in FIG. 5C) made of a semirigid disk in this prototype for clarity, in accordance with various embodiments of the present disclosure.

FIG. 6 is an image showing the shield can be obtained with an inflatable doughnut shaped balloon that is inflated with sterile saline solution only after it is positioned intra-abdominally according to one embodiment of the present disclosure, in accordance with various embodiments of the present disclosure.

FIGS. 7A-7C are images showing an umbrella design of the shield according to one embodiment of the present disclosure. Using a pliable thin material that does not cause intrabdominal adhesions and that can be collapsed when the catheter is introduced in the abdomen and opened when in position, the opening of the device can be obtained using the intrinsic elasticity of the material itself and kept open with slightly more rigid ridges of the same material (like an umbrella), in accordance with various embodiments of the present disclosure.

FIGS. 8A and 8B are images showing a shield PD catheter within the abdominal cavity according to one embodiment of the present disclosure. In FIG. 8A, the shield PD catheter is shown in position with the tip in the Douglas pouch and the shield deployed to create a separation with the rest of the abdominal cavity. In FIG. 8B, the function of the shield is shown preventing the greater omentum to enter in contact with the distal part of the PD catheter where the fluid exchange takes place, therefore preventing its obstruction while the fluid can freely pass around it, in accordance with various embodiments of the present disclosure.

### DETAILED DESCRIPTION

Disclosed herein are various embodiments related to double peritoneal dialysis catheters and peritoneal dialysis catheter shields. Reference will now be made in detail to the description of the embodiments as illustrated in the drawings, wherein like reference numbers indicate like parts throughout the several views.

Peritoneal dialysis (PD) is a home-based therapy for patients with end-stage renal disease (ESRD). The PD processes works by allowing dialysate fluid to flow into the peritoneal cavity via a catheter, where waste products from the blood are filtered and drained out. However, PD catheter (PDC) occlusion remains a significant limitation, often necessitating surgical intervention. The primary mechanism of occlusion is the suction and entrapment of the greater omentum around the openings of a single-lumen catheter. This phenomenon leads to outflow failure and increased healthcare burden. Single-lumen peritoneal dialysis (PD) catheters are susceptible to occlusion by the omentum, which blocks fluid flow and often necessitates surgical replacement, negatively impacting patient outcomes and increasing healthcare costs.

The present disclosure provides an alternative design of a PD catheter to reduce the risk and incidence of malfunction, thus allowing a longer duration and reduced interventions for these patients. The double-lumen catheter of the present disclosure, in some embodiments, includes two intraperitoneal catheter branches that merge into a single extraperitoneal tube. The design reduces the pressure gradient between the lumen of the bifurcated tube and the surrounding omentum, decreasing the suction force on the omentum. The double-lumen catheter exhibits more stable pressure and lower extraction force under partial occlusion, suggesting improved performance in preventing complete blockage.

As used herein, “lumen” refers to a bore of a tube.

The double-lumen catheter maintains more consistent flow even if one lumen is blocked, experiences less negative pressure, and has more consistent suction force, mitigating occlusion risks, thus demonstrating that the double-lumen design may improve dialysis efficiency and the longevity and functionality of peritoneal dialysis catheters in clinical practice. Increasing the diameter of the extraperitoneal tube reduces resistance, leading to more efficient fluid extraction, even when partial occlusion is present.

A shield may be added to the double-lumen catheter to prevent omentum from entering in contact with the distal portion of the catheter where the fluid exchange occurs. The shield creates a barrier between the omentum and the distal catheter portion. The shield can be an inflatable doughnut-shaped balloon or alternatively be an umbrella-shaped design that opens the shield once the shield is in position.

The present disclosure tested, in an ex-vivo model, a novel double-lumen PDC design to enhance flow dynamics and mitigate occlusion risks, ultimately reducing the frequency of catheter failure and associated complications. Performance was evaluated by comparing the fluid flow characteristics, occlusion resistance, and pressure profiles of the double-lumen PDC to those of a traditional single-lumen catheter. The double-lumen catheter exhibited superior performance, particularly under conditions that mimic partial occlusion by the omentum.

### Methods

### Prototype Design

The double-lumen PDC (D-PDC) was designed with two intraperitoneal catheter branches that merge into a single extraperitoneal tube via a “Y” shaped connector. The intraperitoneal branches have the same diameter (3.5 mm) as the single-lumen catheter, and the external portion is also 3.5 mm in diameter. In one experimental variation, the two branches were connected to a larger external tube with a 7 mm diameter to investigate potential improvements in flow dynamics.

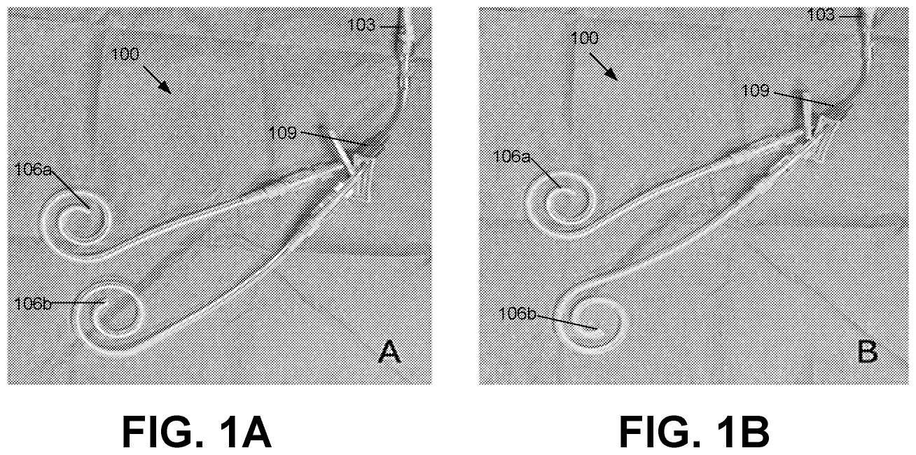

The D-PDC was constructed as a prototype, and experiments were designed to evaluate its performance under controlled laboratory conditions. The catheter and experimental setup are shown in FIGS. 1A-B and 2A-C respectively. In FIGS. 1A and 1B, the two PD catheter (PDC) intraperitoneal terminals (the first 106a and second 106b proximal ends of the split tubing 100) are joined with a “Y” shaped connector (connection point 109) to a single extraperitoneal tube (having distal end 103). In this model, the external single tube is of the same diameter as each PDC (3.5 mm). Alternatively, the two catheters can be joined to a bigger diameter external tube two 3.5 mm to a single 5-7 mm tube. The spiral terminals can be directed in various positions (i.e. illustrated in FIG. 1A: parallel, in FIG. 1B: opposite) and can be of the same length or different lengths, in accordance with various embodiments of the present disclosure.

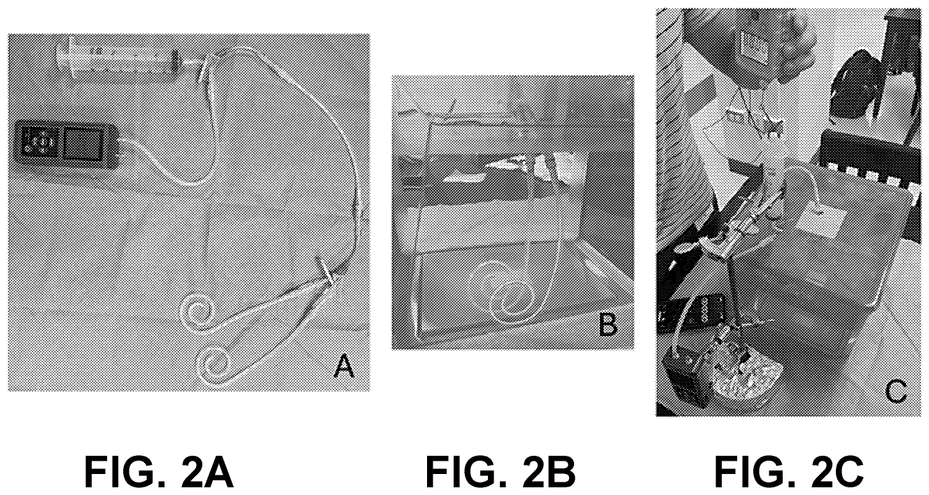

The PDC (either single or double) was connected to a single tube of the same diameter (to mimic the extraperitoneal tube) (shown in FIG. 2A) and submerged in fluid in a closed container to simulate the conditions of the peritoneal cavity with dialysate fluid (FIG. 2B). A 60 cc syringe was used to extract the fluid from the catheter measuring time, volume (mL), force required (Newton (N)), and pressure (Pascal (Pa)) in the system with a dynamometer and a manometer (FIG. 2C), in accordance with various embodiments of the present disclosure.

### Experiment 1: Fluid Extraction Efficiency and Pressure Dynamics

The fluid extraction experiment was set up to simulate the peritoneal cavity filled with dialysate fluid. The D-PDC was submerged in a fluid-filled container. Control measurements were obtained from a standard single lumen PDC in the same experimental conditions and compared to the D-PDC design. A 60 cc syringe was attached to the external catheter end, and fluid was extracted using a standardized pulling force.

The following measurements were recorded: Volume of fluid extracted (ml), Force required for extraction (N) (measured using a dynamometer), and Pressure changes in the system (Pa) (recorded via a manometer).

Three conditions were tested for each catheter design: Nonoccluded (normal condition), Partially occluded (50% blockage), and Fully occluded (for additional tests).

The logic behind testing 50% and 100% occlusion lies in understanding partial versus complete catheter failure. A 50% occlusion mimics early-stage obstruction, where some fluid exchange remains possible, while 100% occlusion represents a worst-case scenario requiring immediate intervention. Fifteen measurements were taken for each condition, and the mean values were calculated for analysis.

### Experiment 2: Effect of Larger Diameter Extraperitoneal Tube

The D-PDC's intraperitoneal branches were connected to a larger 7 mm extraperitoneal tube to assess the impact of tube diameter on fluid flow. Similar tests as in Experiment 1 were conducted, measuring the same parameters. Control measurements were obtained from a standard (3.5 mm) single lumen PDC converting in a wide (7 mm) draining tube in the same experimental conditions and compared to the D-PDC design. This experiment tested whether increasing the diameter of the external tube would reduce the force required for fluid extraction and improve the overall flow rate.

### Experiment 3: Simulation of Omental Occlusion

A human cadaveric greater omentum sample was used to simulate real-life occlusion conditions. The omentum was wrapped around both the single-lumen and double-lumen PDCs. Fluid extraction was performed as in the previous experiments, and measurements for force, pressure, and volume were recorded. This experiment aimed to mimic clinical conditions where the omentum partially blocks the catheter openings, allowing comparison of occlusion resistance between the single and double designs.

### Follow up Study

A follow-up experiment was conducted to further investigate the fluid dynamics associated with different catheter configurations. In this experiment, fluid was drawn using a motor calibrated to a flow rate of 49 mL/h. However, it is important to note that due to the different-sized syringe employed compared to the one for which the machine was originally calibrated, the actual flow rate of fluid drawn did not precisely represent 49 mL/h. Pressure measurements were systematically collected at 5-second intervals over a duration of 60 seconds. This procedure was replicated three times to ensure the reliability and reproducibility of the data.

The experimental conditions included four distinct catheter configurations: (1) a D-PDC with a narrow (3.5 mm) point of convergence, (2) a single PDC (3.5 mm), (3) a D-PDC with a wide (7 mm) point of convergence, and (4) a single PDC with a wide (7 mm) point of convergence. Each configuration was carefully selected to assess the influence of catheter geometry on pressure dynamics. The pressure data were recorded and analyzed to elucidate the differences in performance between the various catheter types under controlled flow conditions.

### Statistical Analysis and Calculations

A two-tailed t-test was performed for each condition to compare the force and pressure values between the single and double PDCs. Results were considered statistically significant at p<0.05.

To determine the pressure differential observed within the D-PDC, calculations were based on the assumption that the flow in the catheter tube is laminar flow. Defining region 1 as the segment of the single tube and region 2 as the bifurcated sections, the relative flow velocity, pressure, and drag force were calculated. By applying Equations 1a and 1b, values were obtained for flow velocity both before and after bifurcation, subsequently substituting these into Equation 2. Assuming a negligible height difference, substituting V₂ into Equation 2 reveals that the pressure in the bifurcated section is greater than in the single tube. This increase in pressure is significant for its potential implications. The higher pressure not only directs flow toward the single tube but also reduces the likelihood of occlusion by the greater omentum. The elevated pressure reduces the pressure gradient between the lumen of the bifurcated tube and the surrounding omentum, decreasing the suction force on the omentum. Using these parameters in Equations 3 and 4, there was a reduced drag force and head loss within the bifurcated tube compared to the single tube.

To derive the pressure, head loss, and force exerted on the flow in the single and bifurcated tube, the Continuity equation (Eqn 1) is employed. With V₁ indicating the flow velocity in the smaller tube and A₁ representing the area of the smaller tube, the flow rate is equal to the flow rate in the larger tube, with V₂ and A₂ representing the larger tube's flow velocity and area, respectively. Then assuming the larger tube's area is double that of the smaller tube, a substitution is made for A₂=2A₁ to determine that V₁=2V₂. Using the area and velocity relationships that were determined in Equation 1, a similar substitution is made in Bernoulli's Equation (Eqn 2), the head loss equation (Eqn 3), and the drag force (Eqn 4). It is determined that the pressure within the bifurcated tube is greater than that in the single tube, and the head loss and drag force experienced in the single tube is greater than in the bifurcated tube.

$$Q = {V}_{1} {A}_{1} = {V}_{2} {A}_{2} = {V}_{2} ( 2 {A}_{1} ) \qquad ( Eqn 1 a )$$

$${V}_{1} = 2 {V}_{2} \qquad ( Eqn 1 b )$$

$${P}_{1} + \frac{1}{2} ρ {v}_{1}^{2} + ρ g {h}_{1} = {P}_{2} + \frac{1}{2} ρ {v}_{2}^{2} + ρ g {h}_{2} \qquad ( Eqn 2 )$$

$${P}_{2} - {P}_{1} = Δ P = \frac{1}{2} ρ {v}_{1}^{2} - \frac{1}{2} ρ {v}_{2}^{2} = 2 ρ {v}_{2}^{2} - \frac{1}{2} ρ {v}_{2}^{2} = \frac{3}{2} ρ {v}_{2}^{2} \qquad ( Eqn 2 )$$

$${P}_{2} > {P}_{1} \qquad ( Eqn 2 )$$

$${h}_{f 1} = f \frac{L {V}_{1}^{2}}{2 {D}_{1} g} = f \frac{L ( 4 {V}_{2}^{2} )}{2 {D}_{1} g} = f \frac{2 L {V}_{2}^{2}}{{D}_{1} g} \qquad ( Eqn 3 )$$

$${h}_{f 2} = f \frac{L {V}_{2}^{2}}{2 {D}_{2} g} = f \frac{L {V}_{2}^{2}}{4 {D}_{1} g} \qquad ( Eqn 3 )$$

$${F}_{D 1} = \frac{1}{2} ρ {v}_{1}^{2} {C}_{D} {A}_{1} = \frac{1}{2} ρ ( 4 {v}_{2}^{2} ) {C}_{D} {A}_{1} = 2 ρ {v}_{2}^{2} {C}_{D} {A}_{1} \qquad ( Eqn 4 )$$

$${F}_{D 2} = \frac{1}{2} ρ {v}_{2}^{2} {C}_{D} {A}_{2} = \frac{1}{2} ρ {v}_{2}^{2} {C}_{D} ( 2 {A}_{1} ) = ρ {v}_{2}^{2} {C}_{D} {A}_{1} \qquad ( Eqn 4 )$$

### Results

In normal conditions, single and double PDC requires similar force to extract the fluid. When the catheter is partially (50%) occluded (simulating the anatomical condition when the greater omentum causes a partial occlusion), there is an increase in the force required to extract the fluid especially in the single catheter because the double PDC can supplement flow with the additional patent catheter.

There is also a greater negative pressure recorded in the partially occluded single PDC (~30-50 Pa) that can cause more suction of omental structures. This is believed to cause a more tenacious adherence of the omentum to the holes of the PDC rendering its occlusion more difficult to reverse with flushing. In other words, this data supports that with the single catheter partially occluded, being the suction pressure higher, the omentum is sucked in the holes and goes deeper in the catheter increasing the risk that it becomes permanently adherent causing the catheter failure.

### Experiment 1: Fluid Extraction and Pressure Dynamics

The double partially occluded catheter, despite requiring slightly more force (compared to non-occluded model) to extract the fluid, has less negative (suction) pressure, that is even lower than non-occluded models. Therefore, this design can reduce the risk of suctioning omental structures during PD fluid extraction.

In the circumstance that this omentum is in proximity with one of the two PDC, this lower suction pressure should prevent omentum to become stuck in the catheter holes. Moreover, if this occurs, it should be less adherent and allowing easier flush out and disengagement of the omentum. These advantages should allow restoring a non-occluded state prolonging the longevity and function of the PDC in the double model in vivo.

This experiment assessed the fluid extraction capacity of single- and double-lumen PDC under normal and occluded conditions. Results showed that the double-lumen catheter exhibited more stable pressure and lower extraction force under partial occlusion, suggesting improved performance in preventing complete blockage.

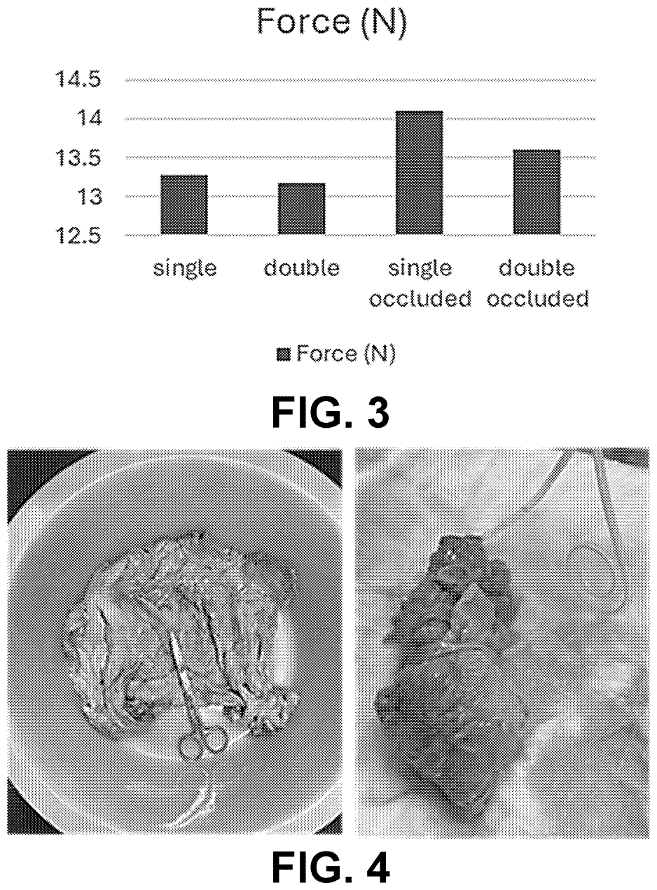

FIG. 3 is a graph with the experimental results. The PD catheter (single) had a measured volume of 23.8 mL, a required force of 13.27 N, and a pressure of 1873.133 Pa. The twin PD catheter (double) had a measured volume of 25.4 mL, a required force of 13.18 N, and a pressure of 1877.467 Pa. The partially occluded PD catheter (single occluded) had a measured volume of 24.6 mL, a required force of 14.10 N, and a pressure of 1906.667 Pa. The partially occluded twin PD catheter (double occluded) had a measured volume of 22.7 mL, a required force of 13.61 N, and a pressure of 1855.813, in accordance with various embodiments of the present disclosure.

The single-lumen PDC exhibited an average fluid extraction volume of 23.8 ml under normal conditions, with a force of 13.27 N and pressure of −1873 Pa. When partially occluded, the single-lumen catheter required a significantly higher extraction force of 14.10 N, and the pressure increased to −1906 Pa (p=0.03363).

In contrast, the D-PDC showed superior performance with an average fluid extraction volume of 25.4 ml under normal conditions. The force required for extraction was slightly lower at 13.18 N, and the pressure was-1877 Pa. When partially occluded, the double-lumen catheter required only a slight increase in force (13.61 N), and the pressure remained relatively stable at −1855 Pa (p=0.07611).

The D-PDC's ability to maintain flow despite 50% occlusion suggests its advantage in delaying complete failure, reducing the need in vivo for catheter replacement. Additionally, the D-PDC maintained more consistent performance, demonstrating lower negative pressure fluctuations and a reduced risk of occlusion compared to the single-lumen PDC.

### Experiment 2: Impact of Larger External Tube Diameter

To evaluate the effect of external tube diameter on fluid flow efficiency, the D-PDC was modified connecting it to a Y connector (3.5 mm to 7 mm) draining in a larger diameter tube to mimic a larger extraperitoneal tube. Findings indicated that increasing the diameter reduced negative pressure and resistance, improving overall dialysis effectiveness. The larger 7 mm external tube significantly improved fluid flow in the D-PDC. The average force required for fluid extraction dropped, and the pressure difference between occluded and non-occluded states was reduced. This suggests that increasing the diameter of the external tube reduces resistance, leading to more efficient fluid extraction, even when partial occlusion is present. Increasing the external tube diameter decreased flow resistance and suction pressure, further enhancing catheter function and potentially extending its lifespan.

### Experiment 3: Simulation of Omentum Occlusion

Using a cadaveric greater omentum, this study simulated real-life occlusive conditions to compare catheter performance. The single-lumen catheter demonstrated higher negative suction pressure, increasing the likelihood of omental entrapment, whereas the double-lumen catheter maintained fluid flow even when partially occluded. The tissue was formalin fixed and therefore was less flexible than omentum in vivo, therefore the findings will possibly underestimate its occlusive capacity. The actual omentum used is shown in FIG. 4, both when in a basin (left) and when wrapped around the PD catheter for the experiment (right). In this experiment, the difference in function and flow between a single PD catheter occluded with omentum were compared to a double PD catheter design with one catheter occluded and one open to show the functional advantage of this design, in accordance with various embodiments of the present disclosure.

When the omentum was wrapped around the catheters, the single-lumen PDC exhibited a sharp increase in negative pressure and extraction force. The suction pressure was approximately 30-50 Pa higher in the occluded single-lumen PDC, indicating that the omentum was being pulled deeper into the catheter openings, increasing the risk of complete blockage.

The D-PDC, however, maintained lower suction pressure and required less force to extract fluid, even when one of the lumens was partially occluded by the omentum. The dual pathways allowed the non-occluded lumen to continue functioning, thereby reducing the risk of complete obstruction.

Following the initial results, pressure measurements were taken at 5-second intervals from a fluid-drawing system utilizing both a D-PDC and a single PDC. This experimental procedure was repeated three times to ensure reliability. Comparative analysis indicated a statistically significant difference in pressure between the un-occluded and occluded conditions for the single catheter, as shown in Table 1. In contrast, the D-PDC did not exhibit a statistically significant variation in pressure, as detailed in Table 2.

To further investigate these findings, the experiment was conducted again using a larger catheter that allowed for the convergence of the bifurcated tubes. Average pressure values and standard deviations for each trial are summarized in Table 3 for the single PDC and Table 4 for the D-PDC. Consistent with the initial trials involving the narrowed convergent catheter, the results from this second series reaffirmed that the single catheter displayed a significant difference in pressure between occluded and un-occluded states. However, the D-PDC, converging into a wider catheter, did not demonstrate a significant change in pressure.

The results confirmed that the D-PDC design mitigates occlusion risk by distributing suction forces more evenly and preventing complete blockage by omental entrapment.

TABLE 1
Single PDC Occluded vs Un-occluded; Narrow
Table 1: Single PDC Occluded vs Un-occluded; Narrow

| | Un-occluded Pressure (Pa) | | | Occluded Pressure (Pa) | | |
|---|---|---|---|---|---|---|
| | Run 1 | Run 2 | Run 3 | Run 1 | Run 2 | Run 3 |
| Avg | 1586.416667 | 1544.25 | 1612.666667 | 1546.583333 | 1561.166667 | 1580.166667 |
| Std | 13.15956847 | 26.63942738 | 19.36413429 | 19.96113648 | 10.35579182 | 42.45389656 |
| T-test | P = 0.019094 | | | | | |

TABLE 2
D-PDC Occluded vs Un-occluded; Narrow
Table 2: D-PDC Occluded vs Un-occluded; Narrow

| | Un-occluded Pressure (Pa) | | | Occluded Pressure (Pa) | | |
|---|---|---|---|---|---|---|
| | Run 1 | Run 2 | Run 3 | Run 1 | Run 2 | Run 3 |
| Avg | 1828.333333 | 1379.583333 | 1458.25 | 1656.25 | 1404.75 | 1480.916667 |
| Std | 5.646130346 | 19.71367008 | 37.18779125 | 27.78856861 | 7.545317633 | 15.36500353 |
| T-test | P = 0.27920787 | | | | | |

TABLE 3
Single PDC Occluded vs Un-occluded
Table 3: Single PDC Occluded vs Un-occluded

| | Un-occluded Pressure (Pa) | | | Occluded Pressure (Pa) | | |
|---|---|---|---|---|---|---|
| | Run 1 | Run 2 | Run 3 | Run 1 | Run 2 | Run 3 |
| Avg | 450.000 | 446.9166667 | 432.3333333 | 450.1666667 | 462.6666667 | 465.5 |
| Std | 2.796101182 | 5.91543948 | 7.900901368 | 4.041451884 | 2.964435661 | 4.337992833 |
| T-test | P = 3.46464E−11 | | | | | |

TABLE 4
D-PDC Occluded vs Un-occluded; Large
Table 4: D-PDC Occluded vs Un-occluded; Large

| | Un-occluded Pressure (Pa) | | | Occluded Pressure (Pa) | | |
|---|---|---|---|---|---|---|
| | Run 1 | Run 2 | Run 3 | Run 1 | Run 2 | Run 3 |
| Avg | 418.9166667 | 499.6666667 | 500.9166667 | 464.0833333 | 470.75 | 469.6666667 |
| Std | 3.629633924 | 16.06426488 | 12.36165871 | 9.848473193 | 2.800162333 | 2.933608802 |
| T-test | P = 0.470349364 | | | | | |

### DISCUSSION

PD is a widely utilized treatment for patients with ESRD, offering the advantages of home-based therapy and fewer hospital visits compared to hemodialysis. However, the long-term success of PD is heavily dependent on the functionality of the PDC, which serves as the access point for dialysate infusion and drainage. One of the most frequent complications that lead to catheter failure is occlusion caused by the greater omentum, a fatty layer of the abdomen that can interfere with the catheter's drainage holes. When the omentum becomes trapped or adheres to the catheter openings, it can block fluid flow, leading to reduced efficiency of dialysis, infections, and ultimately the need for catheter replacement.

### Current Catheter Complications and the Role of the Greater Omentum

The obstruction of PDCs by the greater omentum is a well-documented issue in clinical practice. The omentum's tendency to move freely within the abdominal cavity allows it to contact and obstruct catheter openings during fluid exchanges. Once omental tissue enters the small holes of the PDC, it can become tightly lodged due to the suction pressure created during fluid withdrawal. This blockage often results in partial or complete occlusion, making it difficult to drain the dialysate fluid effectively. The ICD-10-CM code T85.691 (Other mechanical complication of peritoneal dialysis catheter) is used for documenting obstructed PD catheters. This code can be used in clinical studies to track outcomes and compare complication rates between traditional and double-lumen catheter designs. In many cases, such occlusions require mechanical intervention, such as flushing the catheter with saline or repositioning it surgically. If these methods fail, the catheter may need to be replaced entirely, leading to additional surgical procedures and increased risk of complications, including infection and peritonitis.

Studies have shown the lifespan of a PD catheter to be 2-3 years with survival rates over 12, 24 and 36 months at 80.2%, 72.4% and 61.6%, respectively, and failure mostly due to outflow failure such as occlusion by the omentum. Current single-lumen PDC designs exacerbate this problem because they rely on a single pathway for fluid exchange. When this pathway becomes obstructed, there is no alternative route for fluid to flow, leading to poor drainage and potentially life-threatening complications for patients if not corrected promptly.

### Advantages of the Double-Lumen Catheter Design

The goal of this study is to test a novel D-PDC catheter design that enhances flow dynamics and minimizes occlusion risk. By addressing a well-documented failure mechanism—omentum-induced obstruction—this design has the potential to reduce catheter replacements, improve patient outcomes, and lower healthcare costs. If this design is widely implemented, it could transform PD efficiency, making home dialysis more sustainable for patients with ESRD.

The novel D-PDC presented in the present disclosure addresses many of these complications by providing an alternative fluid pathway in the event of partial occlusion. A significant advantage of the double-lumen design lies in its redundancy. Unlike single-lumen catheters, where occlusion of the drainage holes effectively halts fluid flow, the D-PDC ensures that if one lumen becomes obstructed by omental tissue, the second lumen can continue to function. This significantly reduces the risk of complete occlusion, allowing dialysis to proceed with less interruption.

In experiments, the D-PDC required less force to extract fluid compared to the single-lumen PDC, even under partially occluded conditions. The data suggests that the dual lumens provide greater flow stability, as evidenced by the lower negative pressures recorded during fluid extraction. Notably, when the single-lumen catheter was partially occluded, the suction pressure increased substantially (~30-50 Pa), which could draw the omentum deeper into the catheter, making it harder to dislodge and clear. In contrast, the D-PDC maintained a lower suction pressure even under similar occlusive conditions. This lower pressure is advantageous in preventing omental tissue from being sucked into the catheter orifices, reducing the risk of permanent adherence and catheter failure.

Furthermore, the double-lumen design decreases the likelihood of omental entrapment by distributing the suction force more evenly across the two lumens. If one lumen becomes occluded, the second lumen compensates, thereby preventing the excessive negative pressure that typically exacerbates omental entrapment in single-lumen designs. This is particularly important because once the omentum becomes tightly wedged into the catheter openings, it can be difficult to reverse the occlusion, often requiring surgical removal or replacement of the catheter.

### Reduction in Catheter Replacements

The implications of these findings are significant for clinical practice. By reducing the frequency of complete occlusions, the D-PDC design has the potential to reduce the number of catheter replacements required due to omental entrapment.

Currently, catheter replacement surgeries not only increase healthcare costs but also pose significant risks to patients, including infection, extended recovery times, and reduced quality of life. A catheter that is less likely to become occluded will extend the functional lifespan of the device, decrease the need for replacement surgeries, and reduce the overall burden on patients and healthcare systems.

To substantiate the burden of catheter failure, CPT codes for insertion and removal of PD catheters can be referenced:
Insertion: 49421-Placement of a peritoneal dialysis catheter
Removal: 49422-Removal of a malfunctioning peritoneal dialysis catheter

Each catheter failure necessitates a minimum of two additional procedures-removal and reinsertion-exposing patients to additional risks such as infection, surgical trauma, and financial burden. The novel double-lumen design could significantly reduce these occurrences.

The ability to maintain fluid flow in the presence of partial occlusions also has important implications for patient outcomes. Continuous and effective dialysis is crucial for the management of ESRD, and interruptions due to catheter malfunctions can lead to fluid imbalances, inadequate waste removal, and increased morbidity. By ensuring more consistent and reliable fluid flow, the double-lumen catheter design not only enhances the effectiveness of PD but also contributes to better overall health outcomes for patients.

### Economic and Patient Burden of Catheter Failure

The failure of PD catheters due to occlusion, malposition, or infection significantly increases the economic and personal burden on both healthcare systems and patients. Each catheter failure necessitates surgical intervention, leading to additional costs, increased risk of complications, and a decline in patient quality of life. Understanding the financial and clinical impact of catheter failure underscores the need for an improved design that reduces replacement rates and enhances long-term dialysis success.

The financial cost of PDC replacement is substantial, with estimates ranging between $3,000 and $8,000 per procedure when accounting for surgical costs, hospital stays, and follow-up care. These costs accumulate quickly, especially considering that PDC survival rates decline to 61.6% by three years, meaning a significant portion of patients require at least one catheter replacement within that timeframe.

Beyond financial strain, catheter failure directly affects patient comfort and quality of life. Patients undergoing multiple catheter replacements experience pain, longer recovery periods, and psychological stress, reducing adherence to peritoneal dialysis. Furthermore, repeated procedures increase the risk of peritonitis and sepsis, which are among the leading causes of PD discontinuation. By minimizing occlusion-related failures, the proposed D-PDC has the potential to reduce healthcare costs, lower infection risk, and improve patient outcomes by decreasing the number of surgical interventions required.

### Additional Benefits and Future Improvements

The results also suggest that increasing the external tube diameter further enhances the performance of the double-lumen catheter. In the second experiment, when the intraperitoneal double tubes were joined to a larger 7 mm extraperitoneal tube, there was a significant reduction in the force required to extract fluid. This finding indicates that a larger external diameter helps to reduce the resistance encountered during fluid withdrawal, making the process smoother and more efficient.

The double-lumen PD catheter of the present disclosure can ensure dual fluid pathways that maintain flow even in the presence of partial occlusion. Additionally, recent research highlights that occlusion is the leading cause of catheter failure, with omentum-related obstruction accounting for a significant percentage of complications. By distributing suction forces more evenly and reducing negative pressure at the catheter tip, the double-lumen design mitigates omental wrapping, a key failure mechanism. These findings suggest that the present disclosure could substantially reduce replacement rates, lower the risk of peritonitis, and enhance peritoneal dialysis efficiency, making it a valuable advancement in PD catheter technology and leading to better patient outcomes.

### CONCLUSION

The D-PDC presents a significant advancement over traditional single-lumen designs. By providing a dual fluid pathway and reducing the negative suction pressures that contribute to omental occlusion, the present disclosure has the potential to prolong catheter life, reduce the need for surgical replacement, and improve the overall efficiency of peritoneal dialysis. These findings suggest that the double-lumen catheter could play a crucial role in improving outcomes for patients with ESRD, particularly those at risk of recurrent catheter occlusions.

### Shield Designs

Various embodiments of the present disclosure incorporate a shield in the catheter to form a physical barrier between the omentum and the proximal portion of the catheter where the orifices (or openings) are present.

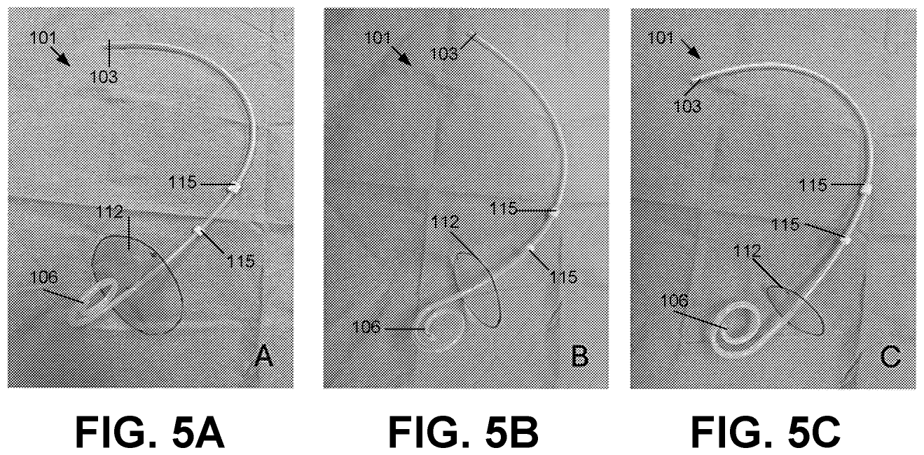

In FIGS. 5A-5C, a shield 112 is shown on a tubing 101 having a distal end 103, a proximal end 106, and a plurality of Dacron cuffs 115 disposed on the tubing 101.

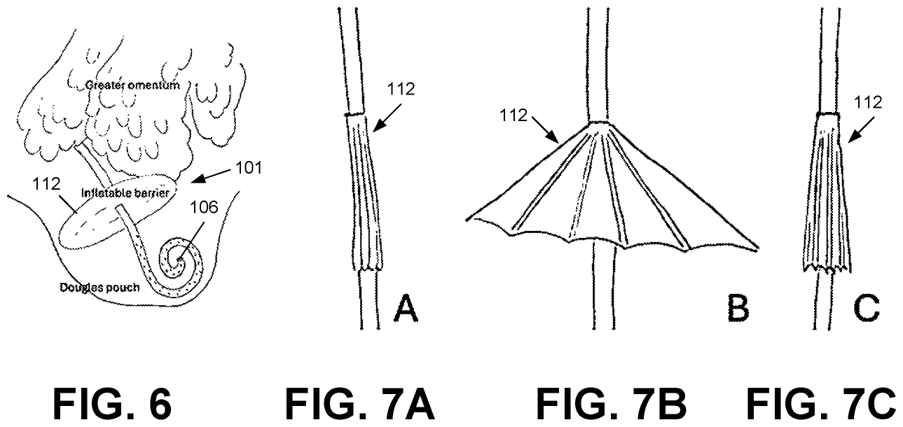

In some embodiments, the shield 112 can be obtained with an inflatable doughnut shaped balloon that is inflated with sterile saline solution only after it is positioned intra-abdominally (FIG. 6). The inflation/deflation can be performed through a separate channel/port on the catheter allowing inflating/deflating the balloon from outside the abdomen.

Alternatively, the same shield 112 concept can be obtained with a different design, i.e. using a pliable thin material (silicon, Dacron or similar) that does not cause intrabdominal adhesions and that can be collapsed when the catheter is introduced in the abdomen and opened when in position. The opening of the device can be obtained using the intrinsic elasticity of the material itself and kept open with slightly more rigid ridges of the same material (like an umbrella) (FIGS. 7A-C).

Other designs can be also created. This shield 112 should be collapsable to facilitate introduction and removal.

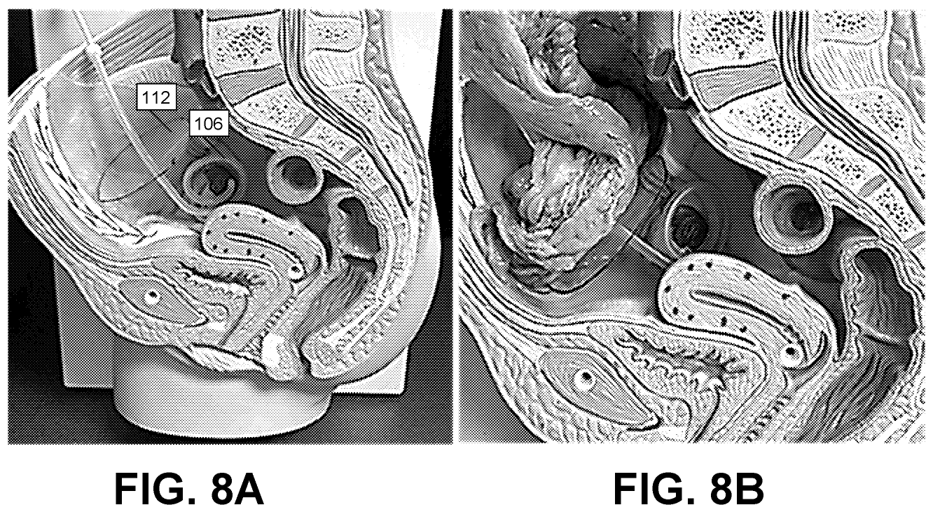

The shield 112 has the purpose of creating a physical barrier preventing the greater omentum to enter in contact with the proximal end 106 of the catheter containing the orifices for fluid exchange that is positioned in the lowest recess of the abdomen called Douglas Pouch (shown in FIGS. 8A-B).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Another embodiment, among others, includes a method, comprising: introducing a catheter device of the present disclosure into an abdominal cavity of a patient, positioning the catheter within a Douglas pouch of the abdominal cavity of the patient, and, in some examples, configuring a shield along the catheter to prevent contact between an omentum of the patient and the proximal end of the catheter.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A catheter device, comprising:
a split tubing comprising a distal end, a plurality of proximal ends comprising a first proximal end and a second proximal end, and a connection point between the distal end and the plurality of proximal ends, wherein the split tubing further comprises:
a primary lumen extending axially through the split tubing from the distal end to the connection point;
a first secondary lumen extending axially through the split tubing from the connection point to the first proximal end of the split tubing;
a second secondary lumen extending axially through the split tubing from the connection point to the second proximal end of the split tubing; and
one or more openings disposed along an external surface of the first and second proximal ends of the split tubing, wherein each of the one or more openings extends transversely through a wall of the split tubing and is in fluidic communication with the first and second secondary lumens, and wherein the first and second secondary lumens are in fluidic communication with the primary lumen of the split tubing at the connection point.

2. The catheter device of claim 1, wherein a diameter of the primary lumen is greater than a diameter of the first secondary lumen and a diameter of the second secondary lumen.

3. The catheter device of claim 1, wherein the first and second proximal ends are coiled.

4. The catheter device of claim 1, wherein the split tubing further comprises one or more Dacron cuffs surrounding an exterior surface of the split tubing and configured to engage a tissue of a user.

5. The catheter device of claim 1, wherein the distal end of the split tubing comprises a connector configured to engage one or more fluid reservoirs.

6. The catheter device of claim 1, further comprising one or more shields configured to engage the external surface of the split tubing between the first and second proximal ends and the connection point, or between the distal end and the connection point, wherein the one or more shields extend radially outward from the external surface of the split tubing.

7. A catheter system, comprising:
a tubing comprising an internal lumen extending axially through the tubing from a proximal end to a distal end, and one or more openings disposed along an external surface of the proximal end, wherein each of the one or more openings extends transversely through a wall of the tubing and is in fluidic communication with the internal lumen; and
a shield configured to engage an external surface of the tubing at a point between the proximal end and the distal end and extend radially outward from the tubing.

8. The catheter system of claim 7, wherein the distal end of the tubing comprises a connector configured to engage one or more fluid reservoirs or a second tubing.

9. The catheter system of claim 7, wherein the proximal end of the tubing is coiled.

10. The catheter system of claim 7, wherein the tubing further comprises one or more Dacron cuffs surrounding the exterior surface of the tubing and configured to engage a tissue of a user.

11. The catheter system of claim 7, wherein the shield is a disk shape.

12. The catheter system of claim 7, wherein the shield is an inflatable donut shaped balloon and the tubing further comprises an inflation channel extending axially from the distal end through the tubing to the balloon and adjacent to the internal lumen, wherein the inflation channel is in fluidic communication with the balloon.

13. The catheter system of claim 7, wherein the shield is a collapsible umbrella shield configured to remain collapsed along the exterior surface of the tubing when introduced in an abdomen of a user and to open away from the exterior surface of the tubing when the tubing is positioned within the abdomen of the user.

14. A method, comprising:
introducing a catheter into an abdominal cavity of a patient, the catheter comprising:
a tubing, an internal lumen extending axially through the tubing, a proximal end, a distal end, and one or more openings disposed along an external surface of the proximal end, wherein each of the one or more openings extends transversely through a wall of the tubing and is in fluidic communication with the internal lumen; and
a shield configured to engage an external surface of the tubing at a point between the proximal end and the distal end and extend radially outward from the tubing, wherein the shield is an inflatable donut shaped balloon and the tubing further comprises an inflation channel extending axially from the distal end through the tubing to the balloon and adjacent to the internal lumen, wherein the inflation channel is in fluidic communication with the balloon;
positioning the catheter within a Douglas pouch of the abdominal cavity of the patient; and
inflating the balloon by injecting a fluid into the inflation channel.

15. The method of claim 14, further comprising:
deflating the balloon by withdrawing the fluid from the inflation channel; and
removing the catheter from the abdominal cavity of the patient.

16. The method of claim 14, wherein the distal end of the tubing comprises a connector configured to engage one or more fluid reservoirs or a second tubing.

17. The method of claim 16, further comprising:
connecting a first fluid reservoir comprising a dialysis solution to the connector;
introducing the dialysis solution into the abdominal cavity through the catheter; and
disconnecting the first fluid reservoir from the connector.

18. The method of claim 17, further comprising:
connecting a second fluid reservoir to the connector;
draining a waste fluid from the abdominal cavity through the catheter into the second fluid reservoir; and
disconnecting the second fluid reservoir from the connector.

19. The method of claim 14, wherein the tubing further comprises one or more Dacron cuffs surrounding the exterior surface of the tubing and configured to engage a tissue of the patient.

20. The method of claim 14, wherein the tubing comprises latex, silicone, polyvinyl chloride (PVC), rubber, or a combination of any thereof.
